(19) United States
(12) Patent Application Publication
(10) Pub. No.: US 2026/0212998 A1
(43) Pub. Date: Jul. 23, 2026

(54) Image processing system for organ segmentation

(71) Applicant: Ever Fortune.AI CO., Ltd., Taichung City (TW)

(72) Inventor: TI-HAO WANG, Taichung City (TW)

(73) Assignee: Ever Fortune.AI CO., Ltd., Taichung City (TW)

(21) Appl. No.: 19/027,922

(22) Filed: Jan. 17, 2025

Related U.S. Application Data
(60) Provisional application No. 63/735,439, filed on Dec. 18, 2024.

(51) Int. Cl. G16H 30/40; G06T 7/10

(52) U.S. Cl. CPC G16H 30/40; G06T 7/10; G06T 2207/10072; G06T 2207/20081; G06T 2207/20084; G06T 2207/20104

(57) ABSTRACT

An image processing system for organ segmentation includes an image processing module, a segmentation module, an image display module, a feedback module, and a retraining module. The image processing module performs a preprocessing process on a medical image to form an input image. The segmentation module performs a prediction process on the input image based on a segmentation model to generate a segmentation image. The image display module displays the segmentation image. The feedback module includes an operation unit and an evaluation unit. The operation unit is configured for a user to manually adjust a segmentation result of the segmentation image to form a manually labeled result, and the evaluation unit scores the segmentation result based on the manually labeled result. When a score is below a threshold score, the evaluation unit generates retraining data. The retraining module retrains the segmentation model based on the retraining data.

### BACKGROUND OF THE INVENTION

### Technical Field

The present invention relates generally to image processing, and more particularly to an image processing system for organ segmentation.

### Description of Related Art

Automated training involves building a system capable of dynamically and automatically updating or fine-tuning a machine learning model without the need for extensive manual intervention. This is particularly important in the medical domain due to the following reasons: ever-evolving data, updating medical knowledge, advances in imaging technology, constantly expanding and changing clinical data, and growing demands for personalization.

In the medical domain, delineating organs out of medical images, such as computed tomography (CT) images, is a crucial preparation. Traditionally, delineation process relies on a professional doctor's manual judgments to delineate organs of a patient manually. The introduction of machine learning models could assist the professional doctor's manual judgments and predict a segmentation result of an organ; however, the segmentation result predicted by the machine learning model may still be inaccurate.

Therefore, how to efficiently improve accuracy of organ segmentation results is an urgent issue.

### BRIEF SUMMARY OF THE INVENTION

In view of the above, the primary objective of the present invention is to provide an image processing system for organ segmentation, improving the accuracy of organ segmentation in medical images.

The present invention provides an image processing system for organ segmentation, including an image processing module, a segmentation module, an image display module, a feedback module, and a retraining module.

The image processing module is signally connected to at least one medical image source and receives at least one medical image from the medical image source. A type of the at least one medical image is one of a plurality of image types including a computed tomography (CT) image type, a magnetic resonance imaging (MRI) image type, and a positron emission tomography (PET) image type. The image processing module performs a preprocessing process on the at least one medical image to form at least one input image

The segmentation module is signally connected to the image processing module and includes at least one segmentation model. The segmentation module is configured to perform, based on the at least one segmentation model, a prediction process on the at least one input image to generate a segmentation result of contours of an organ, and generate, based on the segmentation result and the at least one input image, at least one segmentation image that has the segmentation result and the at least one input image.

The image display module is signally connected to the segmentation module. The image display module displays the at least one segmentation image.

The feedback module is signally connected to the segmentation module and includes an operation unit and an evaluation unit. The operation unit is configured for a user to manually adjust the segmentation result of the at least one segmentation image to form a manually labeled result, and the evaluation unit scores the segmentation result based on the manually labeled result to obtain a score accordingly. When the score is below a threshold score, the evaluation unit sets the manually labeled result and the corresponding at least one input image as retraining data.

The retraining module is signally connected to the feedback module and the segmentation module. The retraining module is configured to receive the retraining data and retrain, based on the retraining data received, the at least one segmentation model to generate a new at least one segmentation model and update the existing at least one segmentation model of the segmentation module.

With the aforementioned design, the segmentation result is adjusted based on the user feedback to form the manually labeled result. When the evaluation unit determines the score is below the threshold score, the retraining data will be generated and the retraining module retrains the segmentation model based on the retraining data, improving the accuracy and efficiency of the segmentation model performing organ segmentation, which efficiently improves clinical value and practicability of the image processing system for organ segmentation.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a block diagram of an image processing system for organ segmentation according to a preferred embodiment of the present invention.

### DETAILED DESCRIPTION OF THE INVENTION

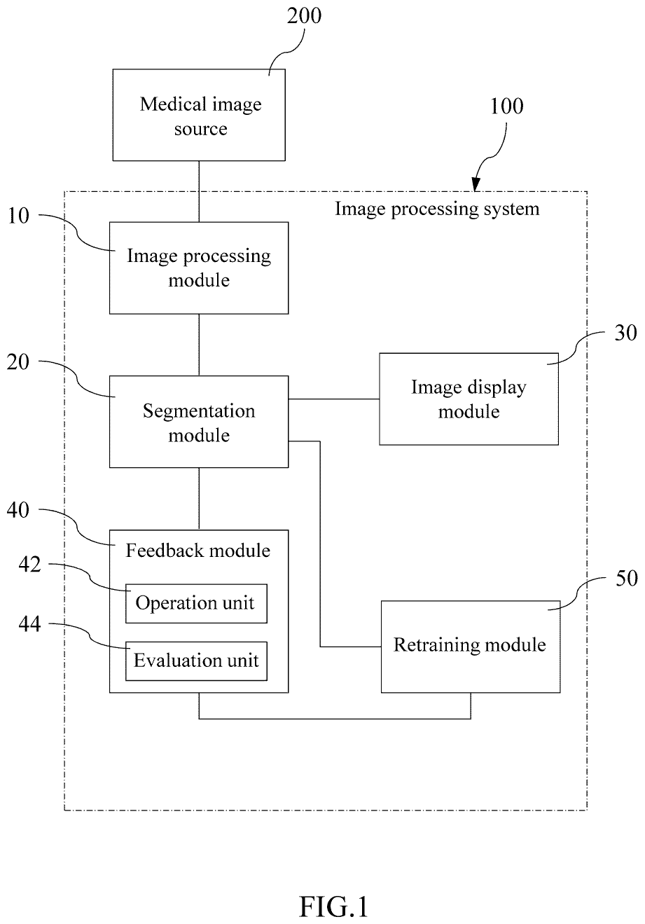

An image processing system 100 for organ segmentation according to a preferred embodiment of the present invention is illustrated in FIG. 1, including an image processing module 10, a segmentation module 20, an image display module 30, a feedback module 40, and a retraining module 50. The image processing system 100 is able to integrate with a Picture Archiving and Communication System (PACS).

The image processing module 10 is signally connected to at least one medical image source 200. The current embodiment focuses on one medical image source 200 as an illustrative example, but it is not limited thereto; the number of the medical image source 200 could be plural. The medical image source 200 could be, for example, a medical imaging equipment, a server, a workstation, or a database. The image processing module 10 communicates with the medical image source 200 based on Digital Imaging and Communications in Medicine (DICOM). The medical image source 200 is configured to provide at least one medical image to the image processing module 10. A type of the at least one medical image is one of a plurality of image types including a computed tomography (CT) image type, a magnetic resonance imaging (MRI) image type, and a positron emission tomography (PET) image type, but the image types are not limited thereto; the image types could further include an X-ray image type, an ultrasound image type, or medical images of other types. The image processing module 10 receives the medical image from the medical image source 200 and performs a preprocessing process to form at least one input image, i.e., to transform the medical image into a format suitable for AI model segmentation and training.

In the current embodiment, the preprocessing process includes image denoising, standardization, and resolution adjustment, ensuring consistency and sharpness across different types of medical images. In addition, given the fact that image contrast and gray levels of medical images may vary from one scanner to another, the preprocessing process further includes image contrast adjustments and gray level standardization so that the image processing module 10 could perform image contrast adjustments and gray level standardization based on properties of medical images. To improve the accuracy of segmentation, the preprocessing process performed by the image processing module 10 further includes enhancement processing techniques, such as boundary enhancement, bone enhancement (for orthopedic images), and specific region enhancement, to make key features in the medical image more visible.

More specifically, the image processing module 10 has a plurality of sets of preprocessing parameters that respectively correspond to the plurality of image types. The image processing module 10 selects one set of corresponding preprocessing parameters based on the type of the at least one medical image, and performs the preprocessing process on the medical image based on the set of preprocessing parameters selected, making the input image generated to correspond to the type of the medical image. In this way, the segmentation module 20 could achieve good performance in performing segmentation on different types of medical images subsequently. For example, each set of preprocessing parameters are optimized parameters required by the preprocessing process performed on each type of the medical images, i.e., each set of preprocessing parameters are parameters required to perform image denoising, standardization, resolution adjustment, contrast adjustment, gray level standardization, and enhancement processing.

The segmentation module 20, signally connected to the image processing module 10, is configured to generate a segmentation result of an organ (i.e., an important structure) in the input image. The segmentation module 20 includes at least one segmentation model. The segmentation module 20 is configured to perform a prediction process on the at least one input image based on the at least one segmentation model to generate the segmentation result of contours of the organ, and generate at least one segmentation image based on the segmentation result and the at least one input image, in which the at least one segmentation image has the segmentation result and the at least one input image.

In the current embodiment, the at least one segmentation model is a pre-trained AI model. The at least one segmentation model employs deep learning techniques, such as convolutional neural network (CNN) or U-Net semantic segmentation model, using a large dataset of preprocessed and annotated medical images to perform a pretraining process so that the segmentation model could, based on the weights in the pre-trained model, perform segmentation on the input image of the image processing module 10 to generate the segmentation result. The segmentation module 20 is characterized by the ability to automatically recognize tissue boundaries, lesions, or specific anatomical structures in the input image and, on this basis, generate high-precision segmentation results. The segmentation module 20 registers the segmentation result and the input image to form the segmentation image.

In the current embodiment, the at least one segmentation model includes a plurality of segmentation models. The plurality of segmentation models respectively correspond to the plurality of image types, i.e., using one corresponding image type and the medical images preprocessed to perform the pretraining process on each segmentation model, thereby each segmentation model capable of predicting the segmentation result based on the input image corresponding to the image type. The segmentation module 20 selects one corresponding segmentation model based on the type of the input image, performs the prediction process on the input image based on the segmentation model selected to generates the segmentation result of contours of the organ. In this way, the segmentation module 20 could automatically select corresponding segmentation model based on the input image to adapt to different imaging features of various image types. Optionally, the segmentation module 20 also supports multiple output formats, allowing the segmentation results generated to be used directly by clinical applications or to be output as a common format for subsequent processing or storage.

The image display module 30, signally connected to the segmentation module 20, is configured to receive and display the at least one segmentation image. In the current embodiment, the image display module 30 is a display device for clinical practice. The user (such as the radiologist or the clinician) inspects the segmentation image on the image display module 30 and could further determine whether the segmentation result generated by the segmentation module 20 is correct or not.

The feedback module 40 is signally connected to the segmentation module 20 and includes an operation unit 42 and an evaluation unit 44. The operation unit 42 is configured for the user to manually adjust the segmentation result of the at least one segmentation image to form a manually labeled result.

For example, the operation unit 42 could be an input device, such as a mouse, a keyboard, or a touch panel, etc. When the user inspects the segmentation image displayed on the image display module 30, the user could directly click on the segmentation result in the segmentation image through the operation unit 42 to perform manual adjustments, such as modifying boundaries delineated, dragging to adjust a specific region, or labeling an incorrect location, to form the manually labeled result that is then recorded by the feedback module 40. In this way, the user could modify the segmentation result in clinical practice based on their own medical expertise, without the need for specific training, facilitating the real-time feedback from the user and enabling the user to operate the operation unit 42 during daily inspection directly.

The evaluation unit 44 scores the segmentation result based on the manually labeled result to obtain a score accordingly. In the current embodiment, the evaluation unit 44 adopts evaluation indicators, such as Dice similarity coefficient, IoU (Intersection over Union), etc., but not limited thereto; they could be other evaluation indicators. Optionally, the evaluation unit 44 could, based on the importance of structures of different organs or the requirements for clinical applications, further assign weights to the evaluation indicators adopted, thereby more accurately reflecting the segmentation quality.

When the score is below a threshold score, the evaluation unit 44 sets the manually labeled result and the corresponding input image as retraining data for subsequent usage when the retraining module 50 retrains the corresponding segmentation model. For example, the score ranges between 0 and 1, and the threshold score could be set as 0.8.

The retraining module 50 is signally connected to the feedback module 40 and the segmentation module 20. The retraining module 50 is configured to receive the retraining data, retrain the at least one segmentation model based on the retraining data received to generate a new at least one segmentation model and update the existing at least one segmentation model of the segmentation module 20.

The retraining module 50 could retrain the segmentation model based on an evaluation result of the evaluation unit 44 and the user feedback, for example, to adjust the weights and parameters of the segmentation model to increase the accuracy and adaptability of the segmentation model, ensuring that the segmentation model could consistently improve and adapt to clinical requirements. In the current embodiment, the retraining module 50 could include an automated learning rate scheduling mechanism that could dynamically adjust the learning rate and weight distribution of the segmentation model based on image feature changes (such as resolution, contrast, structure types of an organ, etc.), ensuring the segmentation model adapts to different situations. In addition, the retraining process could employ incremental learning techniques to enable the segmentation model to learn new data features while preserving prior knowledge.

In the abovementioned, after repeated use of the image processing system 100, the number of the at least one medical image, the at least one input image, and the at least one segmentation image are plural, respectively. In other words. each input image is formed after the image processing module 10 performs the preprocessing process on each medical image, and each segmentation result and each segmentation image are generated after the segmentation module 20 performs the prediction process on each input image, thereby each medical image corresponding to each input image and each segmentation image. The image display module 30 displays each segmentation image.

When the user determines the segmentation result of one of the segmentation images displayed on the image display module 30 needs to be modified, the user manually adjusts the one of the segmentation images to form the manually labeled result. When the user determines the segmentation result of another one of the segmentation images displayed on the image display module 30 is correct, the user sets the segmentation result of the another one of the segmentation images as a correct segmentation state through the operation unit 42.

After the retraining data reaches a predetermined quantity to form a retraining dataset, the retraining module 50 retrains the at least one segmentation model based on the retraining dataset, thereby achieving the objective of updating the at least one segmentation model. In addition, in cases where the segmentation module 20 has multiple segmentation models, the evaluation unit 44 could stratify the retraining data so that the retraining data corresponds to the segmentation model selected by the segmentation module 20. After the retraining data corresponding to any of the segmentation models reaches the predetermined quantity to form the retraining dataset, the retraining module 50 retrains the corresponding segmentation model based on the retraining dataset. In this way, each segmentation model could be retrained respectively.

Optionally, when the structure of the same organ is modified frequently, the retraining module 50 would prioritize the retraining data of the structure of the organ for subsequent retraining to improve the accuracy of the segmentation model in similar cases.

Alternatively, the retraining module 50, at predetermined intervals, determines whether the retraining data reaches the predetermined quantity to form the retraining dataset. When the retraining module, at the predetermined intervals, determines that the retraining data reaches the predetermined quantity to form the retraining dataset, the retraining module retrains the corresponding segmentation model based on the retraining dataset, thereby achieving the objective of regularly updating the at least one segmentation model so that the at least one segmentation model could address the latest image changes and clinical challenges. The predetermined interval could be set according to clinical requirements. In addition, in cases where the segmentation module 20 has multiple segmentation models, the evaluation unit 44 could stratify the retraining data so that the retraining data corresponds to the segmentation model selected by the segmentation module 20. The retraining module 50, at the predetermined intervals, determines whether the retraining data corresponding to any of the segmentation models reaches the predetermined quantity to form the retraining dataset. When the retraining module 50, at the predetermined intervals, determines that the retraining data corresponding to any of the segmentation models reaches the predetermined quantity to form the retraining dataset, the retraining module 50 retrains the corresponding segmentation model based on the retraining dataset. In this way, each segmentation model could be retrained respectively.

The user sets the segmentation result of another one of the segmentation images as the correct segmentation state through the operation unit 42. The operation unit 44 sets the segmentation result corresponding to the correct segmentation state as a standard segmentation result, and the evaluation unit 44 sets the standard segmentation result and one corresponding input image as another retraining data. The retraining module 50 receives the another retraining data and retrains, based on the retraining data received and the another retraining data received, the at least one segmentation model to generate the new at least one segmentation model and update the existing at least one segmentation model of the segmentation module. For example, the retraining module 50 adds the another retraining data to the retraining dataset and retrains the at least one segmentation model based on the retraining dataset that includes the another retraining data. In this way, when the user approves or confirms the segmentation result to be correct, the segmentation result would become a “standard example” of the segmentation model and is set to be the retraining data, which provides high-quality retraining of the segmentation model.

With the abovementioned, the user could seamlessly provide feedback during the daily workflow, and the accuracy of the segmentation model could be continuously optimized through the retraining mechanism of the retraining module 50, which efficiently improves clinical value and practicability of the image processing system 100 for organ segmentation.

It must be pointed out that the embodiment described above is only a preferred embodiment of the present invention. All equivalent structures which employ the concepts disclosed in this specification and the appended claims should fall within the scope of the present invention.

What is claimed is:

1. An image processing system for organ segmentation, comprising:
an image processing module, signally connected to at least one medical image source and receiving at least one medical image from the medical image source, a type of the at least one medical image being one of a plurality of image types including a computed tomography (CT) image type, a magnetic resonance imaging (MRI) image type, and a positron emission tomography (PET) image type; the image processing module performing a preprocessing process on the at least one medical image to form at least one input image;
a segmentation module, signally connected to the image processing module, the segmentation module including at least one segmentation model, the segmentation module configured to:
perform, based on the at least one segmentation model, a prediction process on the at least one input image to generate a segmentation result of contours of an organ;
generate, based on the segmentation result and the at least one input image, at least one segmentation image that has the segmentation result and the at least one input image;
an image display module, signally connected to the segmentation module, the image display module displaying the at least one segmentation image;
a feedback module, signally connected to the segmentation module and including an operation unit and an evaluation unit, wherein the operation unit is configured for a user to manually adjust the segmentation result of the at least one segmentation image to form a manually labeled result, and the evaluation unit scores the segmentation result based on the manually labeled result to obtain a score accordingly; when the score is below a threshold score, the evaluation unit sets the manually labeled result and the corresponding at least one input image as retraining data;
a retraining module, signally connected to the feedback module and the segmentation module, the retraining module configured to:
receive the retraining data;
retrain, based on the retraining data received, the at least one segmentation model to generate a new at least one segmentation model and update the existing at least one segmentation model of the segmentation module.

2. The image processing system for organ segmentation as claimed in claim 1, wherein the at least one segmentation model of the segmentation module includes a plurality segmentation models, the plurality of segmentation models respectively corresponding to the plurality of image types; the segmentation module selects one corresponding segmentation model based on the type of the at least one input image, and the segmentation module performs the prediction process on the at least one input image based on the segmentation model selected to generates the segmentation result of contours of the organ.

3. The image processing system for organ segmentation as claimed in claim 2, wherein the retraining data corresponds to the segmentation model selected; after the retraining data corresponding to any of the segmentation models reaches a predetermined quantity to form a retraining dataset, the retraining module retrains the corresponding segmentation model based on the retraining dataset.

4. The image processing system for organ segmentation as claimed in claim 3, wherein, the retraining module, at predetermined intervals, determines whether the retraining data corresponding to any of the segmentation models reaches the predetermined quantity to form the retraining dataset; when the retraining module, at the predetermined intervals, determines that the retraining data corresponding to any of the segmentation models reaches the predetermined quantity to form the retraining dataset, the retraining module retrains the corresponding segmentation model based on the retraining dataset.

5. The image processing system for organ segmentation as claimed in claim 2, wherein the image processing module has a plurality of sets of preprocessing parameters that respectively correspond to the plurality of image types; the image processing module selects one set of corresponding preprocessing parameters based on the type of the at least one medical image, and performs the preprocessing process on the medical image based on the set of preprocessing parameters selected.

6. The image processing system for organ segmentation as claimed in claim 1, wherein, after the retraining data reaches a predetermined quantity to form a retraining dataset, the retraining module retrains the at least one segmentation model based on the retraining dataset.

7. The image processing system for organ segmentation as claimed in claim 6, wherein, the retraining module, at predetermined intervals, determines whether the retraining data reaches the predetermined quantity to form the retraining dataset; when the retraining module, at the predetermined intervals, determines that the retraining data reaches the predetermined quantity to form the retraining dataset, the retraining module retrains the corresponding segmentation model based on the retraining dataset.

8. The image processing system for organ segmentation as claimed in claim 1, wherein the at least one medical image includes a plurality of medical images, the at least one input image includes a plurality of input images, and the at least one segmentation image includes a plurality of segmentation images; each medical image corresponds to each input image and each segmentation image; the user manually adjusts the segmentation result of one of the segmentation images through the operation unit to form the manually labeled result; the user sets the segmentation result of another one of the segmentation images as a correct segmentation state through the operation unit, the operation unit sets the segmentation result corresponding to the correct segmentation state as a standard segmentation result, and the evaluation unit sets the standard segmentation result and one corresponding input image as another retraining data; the retraining module receives the another retraining data and retrains, based on the retraining data received and the another retraining data received, the at least one segmentation model to generate the new at least one segmentation model and update the existing at least one segmentation model of the segmentation module.
